(19) United States
(12) Patent Application Publication
(10) Pub. No.: US 2006/0009466 A1
(43) Pub. Date: Jan. 12, 2006

(54) Headwear having linear design

(76) Inventor: Min Jong Kim, Kunpo city (KR)

Correspondence Address: DOHYUN PARK;Apt. #2H, 200 Old Palisade road, Fort Lee, NJ, 07024, US

(21) Appl. No.: 10/884,992

(22) Filed: Jul. 7, 2004

(51) Int. Cl. A61K 31/496

(52) U.S. Cl. 514/253.09

(57) ABSTRACT

Headwear having linear design comprises a crown main body having a plurality of panels, a visor portion attached to a forward portion of the peripheral edge of the said crown main body, and protruding fabric lines formed on the said crown portion and visor portion. The said protruding fabric lines consist of the textile fabric forming the crown and the visor portion, and a thin lining material lined the underside of the protruding fabric lines, as well as seam lines formed on both sides of the said fabric line. These protruding fabric lines allow voluminous decoration of headwear, highlighting visually aesthetic effect. A washing treatment of a headwear having these lines renders the lines more clear and conspicuous, thus attracting eyes and further enhancing the product value in effect.

### BACKGROUND OF THE INVENTION

1. Field of the Invention

This invention is related to the field of headwear and, more specifically, to headwear having voluminous fabric lines protruding on the visor and the crown, highlighting visually aesthetic effect.

2. Description of the Prior Art

Baseball style caps, in general, comprises the crown portion, which is the main body of the cap, having a plurality of panels, the visor portion attached to a forward portion of the peripheral edge of the said crown portion, the sweatband attached to the lower part of the inside of the crown, and the size adjuster attached to an underside of the rear part of the crown.

The headwears are widely used day to day by people regardless of their gender and age. Furthermore, the users have come to use headwear not only as gear to merely block sunlight but also as a fashion item to express themselves.

Such consumer needs have been answered by development of an ever-growing number of various shaped crown body, visor portions, sweatband portions and size adjusters.

Despite these efforts to alter the shape of headwear, from the viewpoint of an average user who considers headwear as a fashion item, one can never have enough improvement. The crown body and visor portion, in particular, traditionally mere combinations of a plurality of panels, require more improvement.

### SUMMARY OF THE INVENTION

In view of the foregoing, the objective of the present invention is to provide an improved cap having voluminous lines on the crown body and visor portion of cap, highlighting visually aesthetic effect, and thereby raise the product value of the cap.

In accordance with this objective, a typical headwear item designed using this invention has a crown main body having a plurality of panels, a visor portion secured to a forward portion of the peripheral edge of the said crown main body, and fabric lines protruding on the crown portion and visor portion. The said protruding fabric lines consist of the textile fabric forming the crown and the visor portion, and a lining material lined on the underside this fabric, as well as seam lines on both sides of such fabric lines. This lining material, as occasion demands, may be excluded from the said fabric lines. A washing treatment may render the said protruding fabric lines more clear and conspicuous.

The said protruding fabric lines may be used for any other headwear of any sort than baseball caps.

### BRIEF DESCRIPTION OF THE DRAWINGS

FIG. 1 is a perspective view of a cap using this invention.

FIG. 2 is a perspective view of the textile fabric forming the visor portion, linearly designed using this invention.

FIG. 3 is a sectional view of the textile fabric of the visor portion using this invention.

FIG. 4 is a perspective view of a sun visor shown as an alternative embodiment of this invention.

### DESCRIPTION OF THE PREFERRED EMBODIMENT

In describing preferred embodiments of the invention illustrated in the drawings, it is to be understood that these embodiments are given by way of illustration only. It is not intended that the invention be limited in its scope to the details of construction and arrangement of components set forth in the following description or illustrated in the drawings. Also, in describing the preferred embodiments, specific terminology will be resorted to for the sake of clarity. It is to be understood that each specific term includes all technical equivalents, which operate in a similar manner to accomplish a similar purpose.

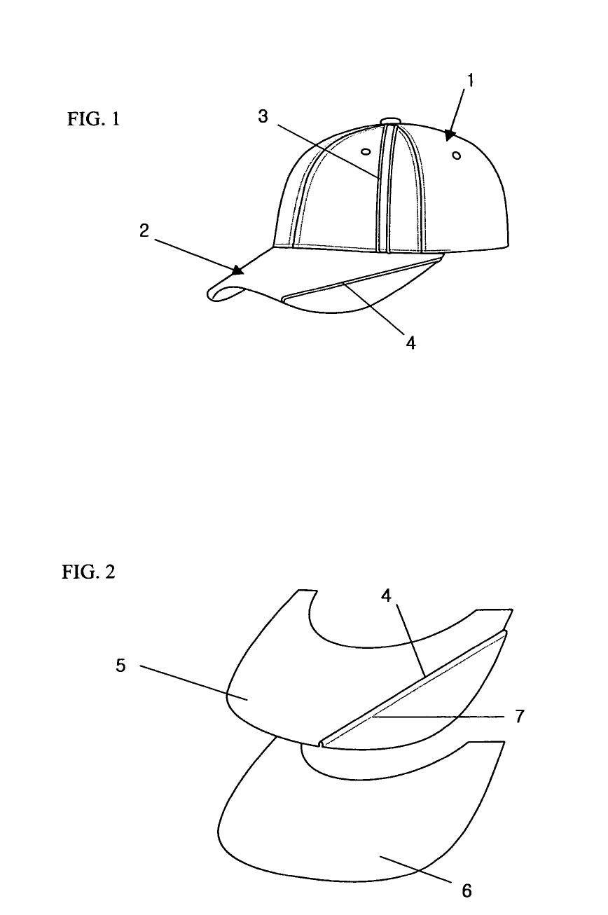

FIG. 1 is a perspective view of a cap using this invention. As shown in FIG. 1, headwear having linear design in this invention comprises the crown portion 1, which is the main body having a plurality of panels, the visor portion 2 attached to a forward portion of the peripheral edge of the said crown main body, and the size adjuster(not visible) attached to an underside of the rear part of the crown.

Formed on the said crown portion and visor portion are protruding and voluminous fabric lines 3 and 4. The above fabric lines 3 and 4 are basically the fabrics forming the crown portion 1 and the visor portion 2, respectively, and have the consistent thickness in appearance. In addition, the said lines consist of the fabric forming the crown portion and the visor portion, the lining material lined the underside of said fabric, and the seam lines formed on both sides of such fabric line. The above lining material lined the underside of the fabric may be excluded from the makeup of the said fabric lines, as occasion demands. The said fabric lines visually appear to the user as voluminous fabric lines and thereby have aesthetic effect. Additionally, the said fabric lines may be presented in various shapes other than straight lines. Moreover, a washing treatment applied to headwear having such fabric lines would decolorize the protruding fabric lines and making the lines more clear and conspicuous, thus attracting eyes and further enhancing the product value in effect.

FIG. 2 is a perspective view of the textile fabric forming the visor portion with linear design and, FIG. 3 is a sectional view of the textile fabric of the visor portion in this invention. As shown in FIGS. 2 and 3, lined the underside of the fabric forming the visor portion in this invention is a lining material 6, and formed above the fabric is protruding and voluminous fabric line 4. Additionally, formed on both sides of the said protruding fabric lines are seam lines 7. The cross section of the said fabric lines has a circular shape. The diameter of the fabric line 4, which may be deemed to as the thickness, is limited to 1 to 3 mm. The said fabric line 4 may be presented in various shapes other than straight lines, depending on the intention of the user.

To make the said protruding fabric line 4 on the visor portion, a thin lining material 6 is lined the underside of the fabric 5 forming the visor portion, and then the said fabric 5 is folded inward to form the protruding fabric line 4. Then, the outer surface of the folded fabric is sewn with sewing thread, leaving a gap of intended thickness in-between. Subsequently, unfolding the folded fabric back forms a fabric line of the consistent thickness. Optionally, the lining material placed on other parts than the said protruding fabric line 4 may be ripped off down the seam lines 7. The protruding line on the crown body may be made in the same manner as on the visor portion. The adequate thickness of the said protruding fabric line is 1 to 3 mm. As mentioned above, the said lining material may optionally be accepted from the manufacture of the protruding fabric line.

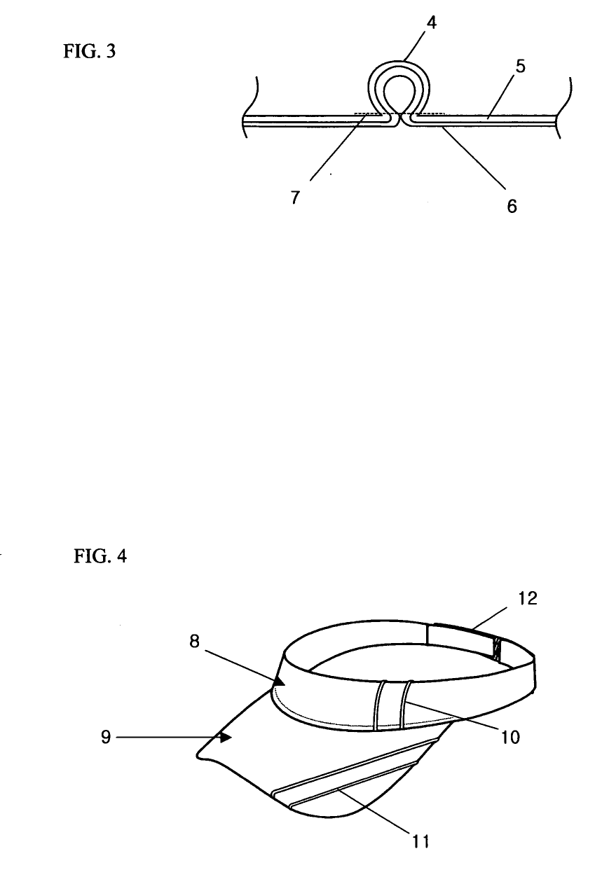

FIG. 4 is a perspective view of the visor shown as an alternative embodiment of this invention. As shown in FIG. 4, this embodiment illustrates that this invention may be applicable to any other types of headwear than baseball-style caps. The visor alternatively embodied here comprises a rim part 8 having a plurality of panels, a visor portion 9 attached to a forward portion of the peripheral edge of the rim part 8, and the size adjuster 12 attached to the rear part of the rim 8. As embodied in the above baseball cap, the protruding fabric lines 11 on the visor portion are basically the fabric forming the visor portion and have the consistent thickness in appearance. In addition, the said line 11 consists of the fabric forming the visor portion, the lining material lined the underside of such fabric, and the seam lines formed on both sides of such fabric line.

The foregoing descriptions and drawings should be considered as illustrative only of the principles of the invention. The invention may be configured in a variety of shapes and sizes and is not limited by the dimensions of the preferred embodiment. Numerous applications of the present invention will readily occur to those skilled in the art. For example, the headband may be incorporated into hats, caps and visors of other styles, or may be used alone. Therefore, it is not desired to limit the invention to the specific examples disclosed or the exact construction and operation shown and described. Rather, all suitable modifications and equivalents may be resorted to, falling within the scope of the invention.

What is claimed is:

1. A baseball style cap having linear design comprising:
a crown main body having a plurality of panels;
a visor portion attached to a forward portion of the peripheral edge of the said crown main body;
protruding fabric lines having a circular sectional shape and a consistent thickness on the fabric forming the said crown body and said visor portion, a thin lining material lined the underside of the protruding fabric line, and seam lines on both sides of the said protruding fabric line. The diameter of the circular sectional shape of the said fabric line is limited to 1 to 3 mm.

2. The headwear as set forth in the claim 1, wherein an optional washing treatment may be applicable to the crown body and visor portion having protruding lines.

3. A headwear comprising:
a crown main body having a plurality of panels;
a visor portion secured to a forward portion of the said peripheral edge of the said crown main body;
protruding fabric lines having a circular sectional shape and a consistent thickness on the fabric forming the said crown body and said visor portion, and seam lines on both sides of said protruding line.

4. The headwear as set forth in claim 3, wherein a thin lining material is attached the underside of the protruding fabric line.

5. The headwear as set forth in claim 3, wherein a washing treatment is applied to the crown main body and visor portion having protruding lines.

6. The headwear as set forth in claim 3, wherein the diameter or the thickness of the protruding fabric line is 1 to 3 mm.

7. A visor comprising:
a rim part having a plurality of panel, a visor portion connected to a lower edge of a front part of the said rim part;
protruding fabric lines having a circular sectional shape and a consistent thickness on the fabric forming the said rim part and the said visor portion, a thin lining material lined underneath the protruding fabric line, and seam lines on both sides of the said protruding line. The diameter of the said fabric line is limited to 1 to 3 mm.

8. The visor as set forth in claim 6, wherein a washing treatment is applied to the rim part and visor portion having protruding lines.
